(19) United States
(12) Patent Application Publication
(10) Pub. No.: US 2006/0206045 A1
(43) Pub. Date: Sep. 14, 2006

(54) Post operative knee brace with multiple adjustment features

(75) Inventors: Jeffrey H. Townsend, Bakersfield, CA (US); Steven S. Knecht, Bakersfield, CA (US)

Correspondence Address: ROBERTS, MLOTKOWSKI & HOBBES, P. O. BOX 10064, MCLEAN, VA, 22102-8064, US

(73) Assignee: Townsend Industries, Inc., Bakersfield, CA (US)

(21) Appl. No.: 11/073,737

(22) Filed: Mar. 8, 2005

(51) Int. Cl. A61F 5/00

(52) U.S. Cl. 602/26; 602/5; 602/16; 602/23

(57) ABSTRACT

A knee brace having adjustment mechanisms that are easy and economical to produce while being very user friendly, and which is more comfortable to wear. The brace has a joint mechanism formed of a plurality of plates with notches, openings and range of motion surfaces, the relative positions of which are used as part of a flexion-extension stop arrangement and also as part of an adjustable locking arrangement by which the brace can be locked, temporarily released or indefinitely released. Furthermore the lateral-medial angulation of the femoral strut is able to be adjusted relative to the joint mechanism and tibial strut.

### BACKGROUND OF THE INVENTION

1. Field of the Invention

The invention relates to orthopedic knee braces and in particular to post-operative knee braces as are commonly applied to the leg of a patient that is convalescing from knee surgery to enable the leg to be rehabilitated without reinjuring of the knee. Such knee braces routinely feature two rigid bars, hinges that can be adjusted to fix the brace so as to preclude flexing of the knee or to limit the extent to which the patient can extend and/or flex the leg. Typically, straps are provided for securing the brace to the leg and pads act to provide a comfortable interface between the bars and hinges and the patient's leg.

2. Description of Related Art

Numerous examples of knee braces of the initially mentioned type and hinges for such knee braces can be found in the art. U.S. Pat. Nos. 4,463,751; 4,982,732; 5,000,169; 5,105,805; 5,409,449; 5,460,599; and 5,814,000 are representative of post-operative knee braces and motion limiting knee hinges. Most range of motion limiting hinges merely enable end limits to the degree of flexion/extension to be set and some also enable the leg to be locked in a fixed position; in the case of U.S. Pat. No. 5,409,449, the hinge mechanism of the brace is provided with a detent mechanism having the ability to release the lock to enable bending of the joint and the ability to automatically relock on straightening of the leg. However, all of the known adjustment mechanisms have been complex and not very user friendly.

It is also noted that, hip braces which are securing in the pelvic region have been provided with medial-lateral angular adjustability; see, e.g., U.S. Pat. No. 5,421,810. However, a need to be able to adjust the angle of the upper bar relative to the knee joint and lower bar in medial and lateral directions has not been found to exist up to this point.

### SUMMARY OF THE INVENTION

Thus, it is a goal of the present invention to provide a knee brace that will have adjustment mechanisms that are easy and economical to produce while being very user friendly. It is a further goal of the present invention to provide a knee brace that will be more comfortable to wear.

The first-mentioned goal is achieved in accordance with present invention using a joint mechanism formed of a plurality of plates with notches, openings and range of motion surfaces, the relative positions of which are used as part of a flexion-extension stop arrangement and also as part of an adjustable locking arrangement by which the brace can be locked, temporarily released or indefinitely released.

Contributing to attainment of the second goal, is a means for enabling the lateral-medial angulation of femoral strut to be adjusted relative to the joint mechanism and tibial strut.

In addition to the foregoing, various objects, features and advantages of the present invention will become apparent from the following detailed description of the invention when viewed in light of the accompanying drawings.

### BRIEF DESCRIPTION OF THE DRAWINGS

FIG. 1 is a perspective view of a knee brace in accordance with the invention on a patient's leg;

FIG. 2 is a frontal view of a unit comprised of the struts and joint from one side of the knee brace of FIG. 1;

FIG. 3 is a side elevational view of the unit of FIG. 2;

FIG. 4 is an exploded view of the unit shown in FIGS. 2 & 3;

FIG. 5 is a view corresponding to that of FIG. 2 with the unit rotated 180° and showing a contouring hinge modification of the tibial strut;

FIGS. 6 & 7 show the lower and upper length adjusting strut segments;

FIG. 8 is a view corresponding to that of FIG. 5, but showing the struts in a fully lengthened position.

FIGS. 9-11 are side views of the FIG. 5 unit showing the tibial strut in laterally angled, neutral and medially angled positions;

FIG. 12 is an exploded view of the hinge of the tibial strut of FIGS. 9-11,

FIGS. 13 & 14 show unlocked and locked positions respectively of a locking lever of range-of-motion adjustment discs,

FIGS. 15 & 16 show unlocked and locked positions respectively of a drop lock mechanism for releasing and fixing the knee joint,

FIGS. 17 & 18 are exploded views of the drop lock mechanism of FIGS. 15 & 16.

FIG. 19 is an enlarged perspective view of a portion of the tibial side area of the joint mechanism with a modified locking lever and extension and modified flexion stop plates, structure of the joint mechanism which covers the stop plates being omitted; and

FIG. 20 is a perspective view of the portion shown in FIG. 19 with the locking lever in its locked position and the brace in a 90° flexed position.

### DETAILED DESCRIPTION OF THE INVENTION

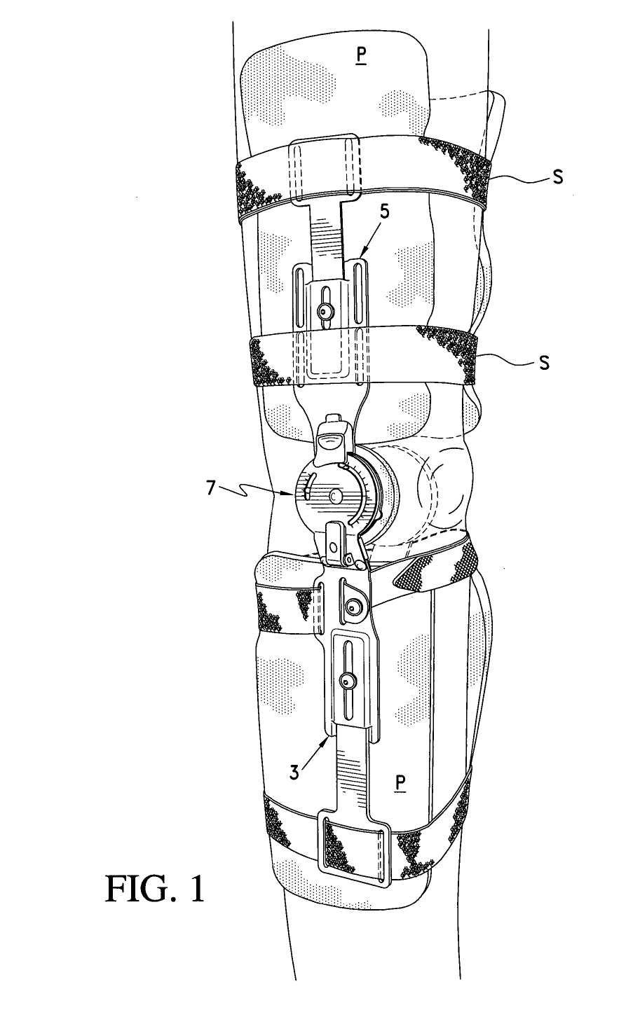

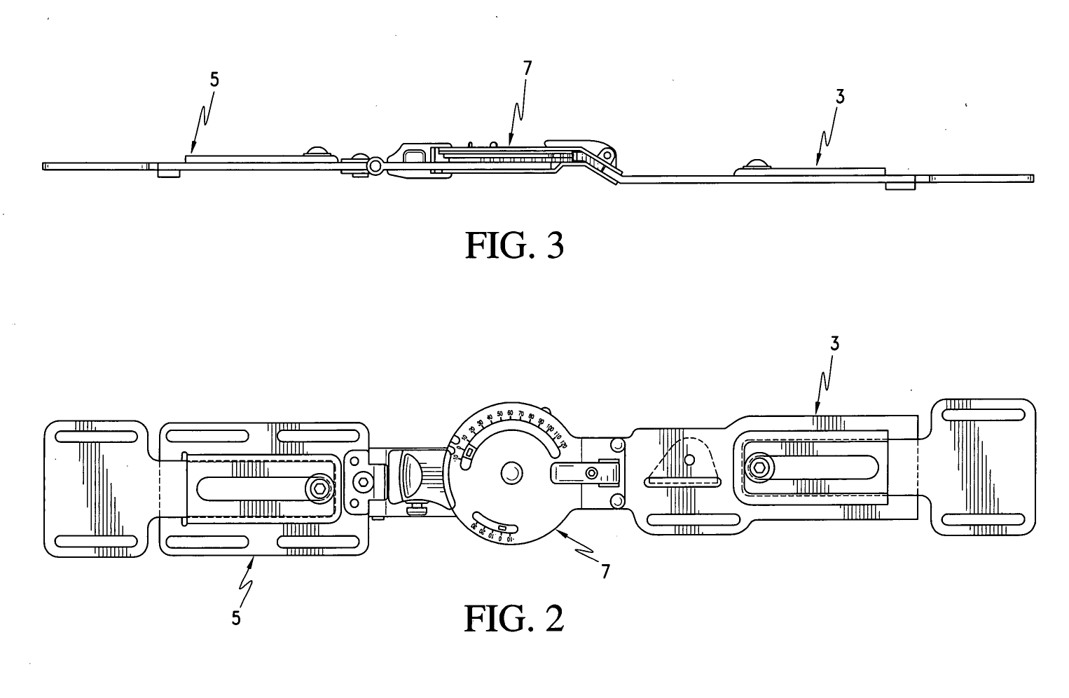

FIG. 1 shows a post-operative knee brace 1 in accordance with the present invention, and while only one side of the brace 1 is shown and described below, it is to be understood that identical structure is present on the opposite side of the brace 1 in mirror-image fashion. Thus, each side of the brace 1 has adjustable length tibial (lower) and femoral (upper) struts 3, 5, respectively. Each tibial strut 3 is connected to each femoral strut 5 by a joint mechanism 7. For attachment to the leg of the patient, a series of straps S are attached to the struts 3, 5 by being passed through slots in the struts and/or being attached to the struts via a fastener. Additional, for comfort purposes, pads P are provided on the inner sides of the struts 3, 5. To this extent, the brace 1 conforms with known knee braces.

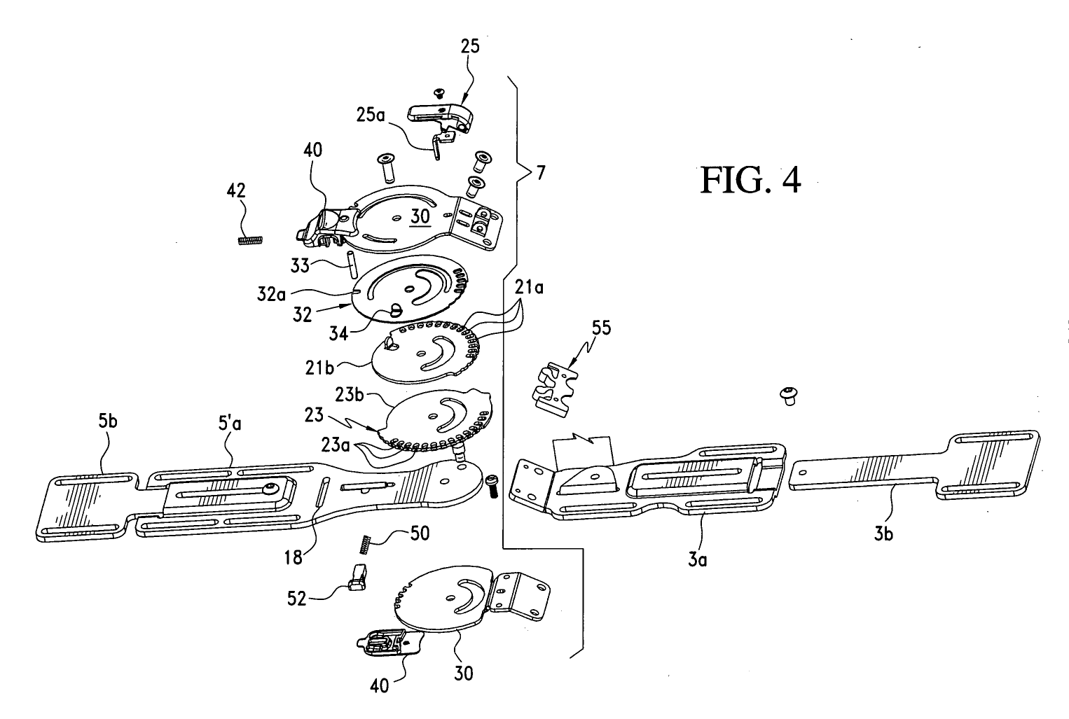

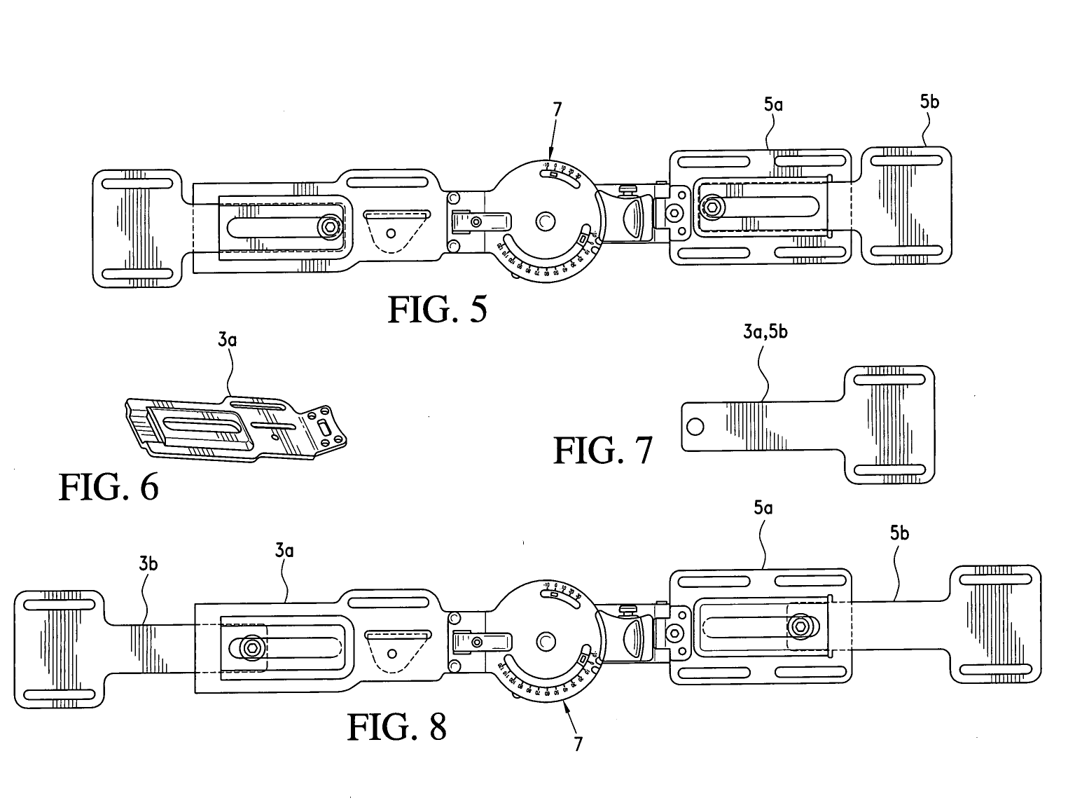

As can be seen most clearly in FIGS. 4-8, the adjustable length struts 3, 5 are comprised of a joint connector part 3a, 5a and an extension part 3b, 5b. The extension parts 3b, 5b are inserted into a pocket slot recess arrangement and are secured in place at a desired relative position (strut length) by a screw 9 that is inserted through the joint connector part 3a, 5a and threaded into the extension part 3b, 5b. The pocket slot recess arrangement, besides providing guidance for adjustment of the extension part 3b, 5b relative to the joint connector part 3a, 5a, also provides the added benefits of increasing the strength of the strut parts and of providing as low a profile as possible.

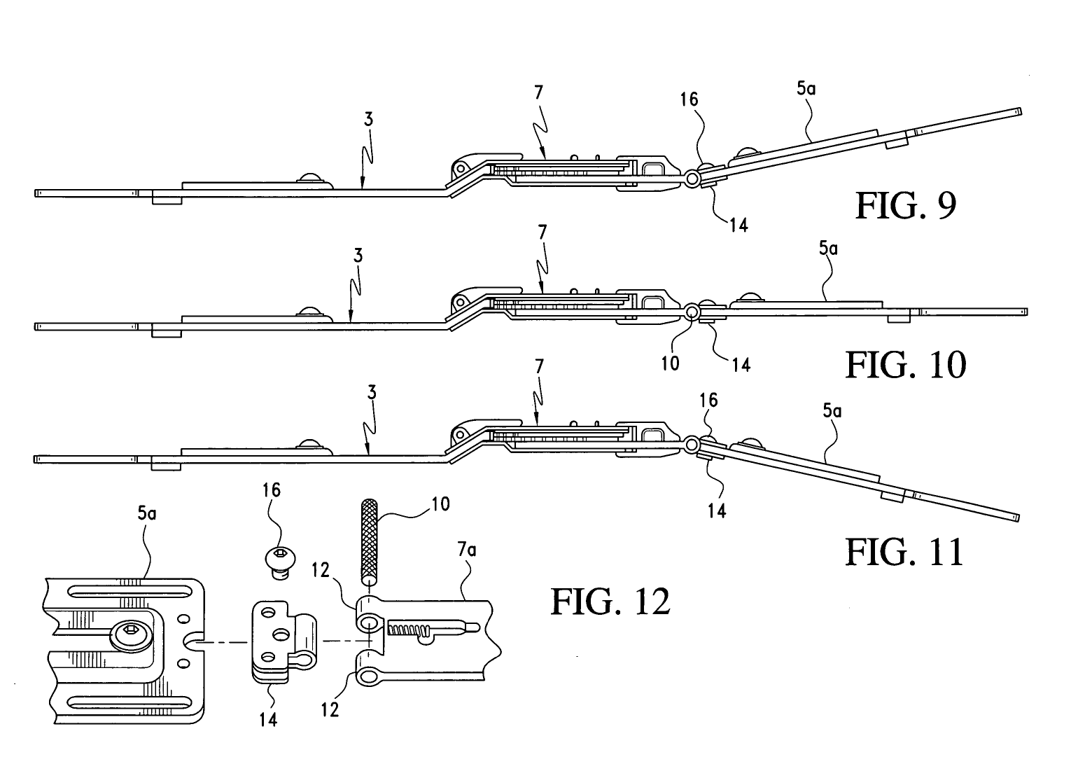

In accordance with a first feature of the invention, shown in FIGS. 9-12, the angle of the femoral bar 5 relative to the joint 7 and tibial bar 3 can be adjusted and then fixed. In particular, a knurled pin 10 is press-fit in eyelets 12 on the lower end of the joint connector part 5a so that it is not free to rotate in the eyelets 12. The knurled pin also passes through a locking clamp 14 which is riveted onto the upper end of the strut connector 7a of the joint mechanism 7 and that can be spread apart or drawn together by a locking screw 16. When the screw is loosened, the knurled pin is free to rotated within the clamp so as to enable the angle of the femoral strut to be changed relative to the joint 7 and tibial strut 3, after which tightening of the screw holds the knurled pin tight within the clamp so that no further relative movement is possible.

A simpler and less costly alternative for obtaining adjustment of the angle of the femoral strut 5 relative to the joint mechanism 7 and tibial strut 3 can be seen in FIG. 4. In particular, in this version, instead of the hinge type arrangement described in the preceding paragraph, the strut connector 7a of the joint mechanism 7 and the joint connector part 5a are integrated into a single one-piece component 5′a, a slot 18 being formed at a location corresponding to that of pin 10 of the FIG. 9-12 version. The slot 18 forms a weakened area which allows the component 5′a to be bent about its centerline by placing the slot 18 over the edge of a table and applying pressure above and below it.

With either version, the brace can be better matched to the contour of the patient's thigh in medial-lateral directions. Where cost is a primary factor and it is not expected that readjustment or fine adjustments will be required, the version with slot 18 will be preferred. On the other hand, where cost is not a primary factor and/or it is expected that the brace may be reused by other patients, so that the need for significant readjustment can be expected, then the version shown in FIGS. 9-12 may be preferable. Furthermore, either of these versions can be use with or without any of the other features described below.

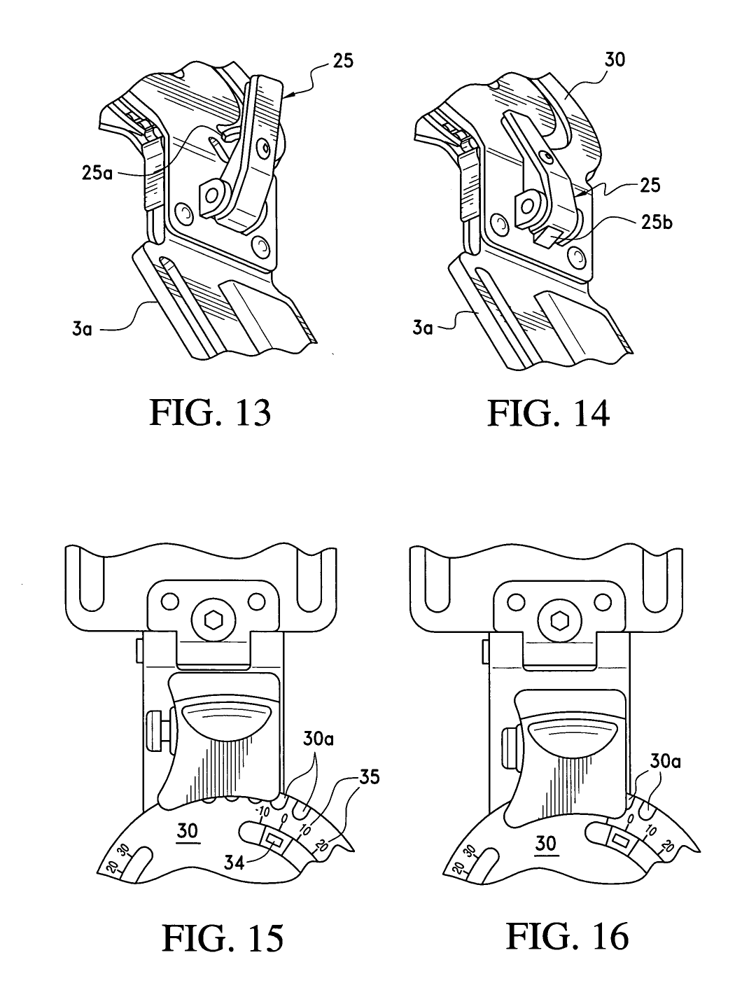

Numerous knee braces are provided with flexion and/or extension stops, as noted in the Background portion of this application and many those braces use joint mechanisms formed of two or more plates; see, e.g., the above-mentioned U.S. Pat. Nos. 4,463,751 and 5,105,805. Likewise, the flexion and extension stop feature of the present invention is formed by a joint mechanism 7 being comprised of a plurality of plates one of which is an extension stop plate 21 and another of which is a flexion stop plate 23, and both of which are provided with a plurality openings 21a, 23a along a portion of their periphery (see, FIG. 4). To set the flexion and extension ranges of motion (ROM), the rotational angle of the respective plate is set at the appropriate point with the locking lever 25 in the position shown in FIG. 13. When the plates are in the proper position, the locking lever 25 is moved to its position shown in FIG. 14, in which a fixing pin 25a, that is provided on the underside of the locking lever 25, is inserted into the respective opening(s) 21a, 23a of the extension and flexion stop plates, thereby fixing their rotational position relative to the struts 3, 5. The range of motion is set by the position of the ROM reduced diameter portions 21b, 23b of the extension and flexion plates 21, 23, and the radial stop surfaces formed at their ends, which are engaged by the latch pin 33 when the set maximum extension and/or flexion is reached.

Of particular significance is the resilient extension 25b formed on the distal side of the pivot from the free end of the lever 25. This extension is shaped so that, in the locked position of FIG. 14, it will hold the lever in that position. However, it is also shaped so that, when force is applied to the lever to lift it, the resilient extension 25b will cam inward so as to permit the locking lever to be lifted. Then, in the open position of FIG. 13, it functions in the manner of a leaf spring to hold the locking lever in the raised open position.

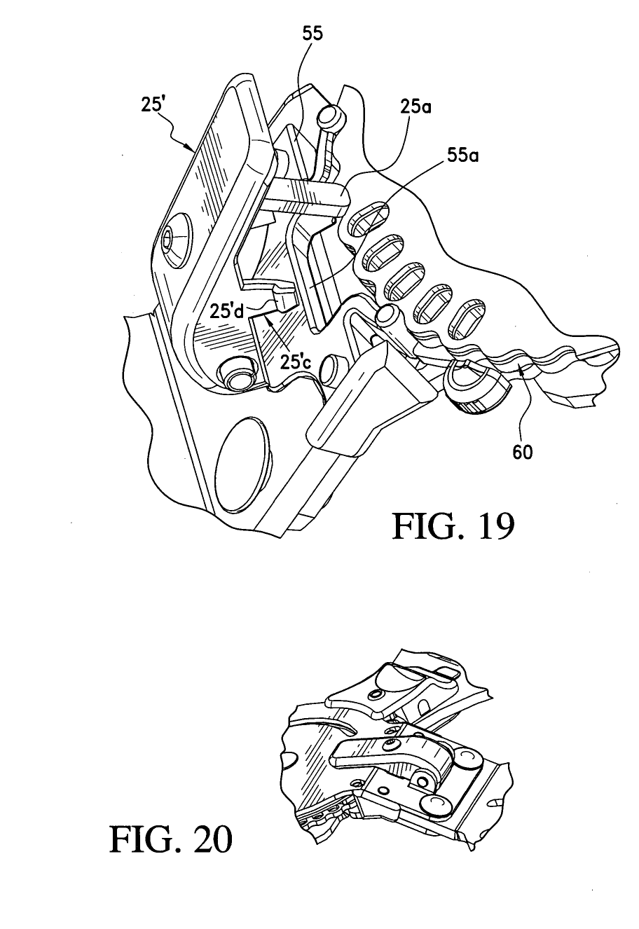

However, by appropriate setting of the tolerances in the sizing of the locking lever 25 and the plate 30 (and/or the pin on which lever 25 pivots), frictional forces can be made sufficient to hold the locking lever 25 in its raised, unlocked position. In such a case, it becomes necessary only to provide a means for holding the locking lever 25 in its locked position. FIG. 19 shows an alternative locking lever 25′. The locking lever 25′ lacks the resilient extension 25b shown in FIG. 14, and instead, has a resilient finger 25c which projects outward at each side. The fingers 25c have detent projections 25d at their ends which clip within a notch 55a of a retainer plate 55 that is attached on the upper end of the tibial strut 3. The inner edge of the detent projections 25d is beveled so that pulling up on the locking lever 25′ will cause them to cam inward and disengage. Likewise the outer end is also beveled to facilitate insertion of the fingers 25c through notch 55a, after which the resiliently spring outward and engage the retainer plate 55.

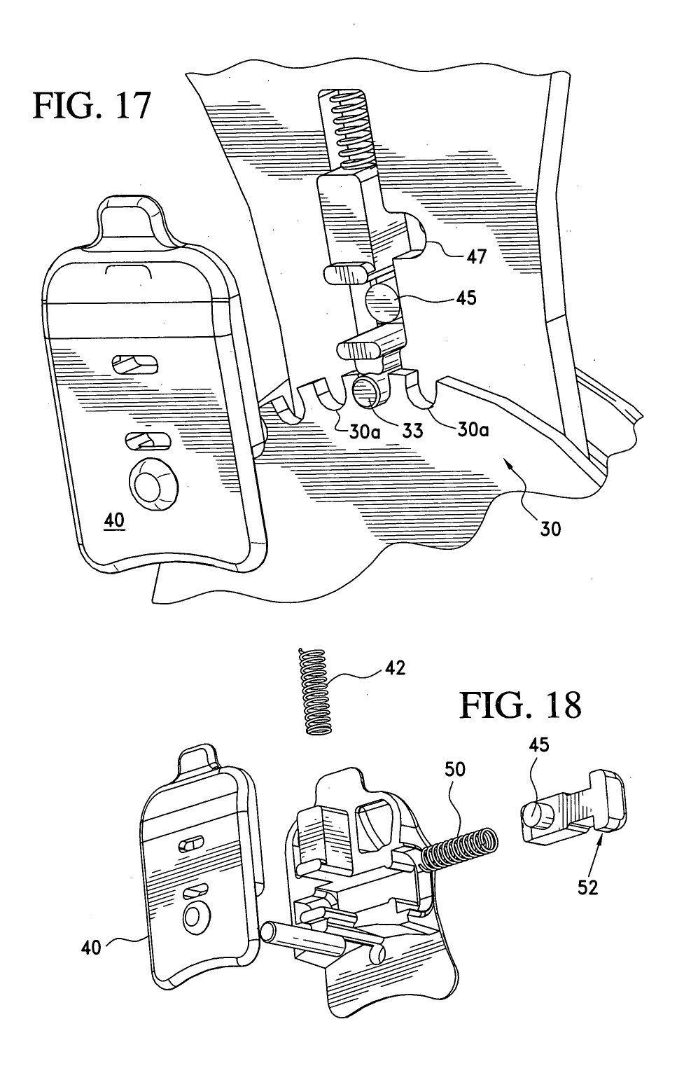

Another feature of the present invention an adjustable locking arrangement that allows the brace to be locked in any of a number of different angular positions, released to allow the knee to bend, e.g., for sitting, and then, lock back into the preset position. With reference to the exploded view of FIG. 4 and FIGS. 15-17, it can be seen that a locking plate 30 has a plurality of peripheral notches 30a while an adjustment lock plate 32 has only one peripheral notch 32a, all of which fall within the ROM reduced diameter portions 21b, 23b of the extension and flexion plates 21, 23. As can be appreciated, the notch 32a in the adjustment lock plate 32 can be rotated so as to be aligned with one of the plurality of notches 30a in the locking plate 30 to lock the femoral and tibial struts 3, 5 in axial alignment (O° position in which the struts form an included angle of 180°) or in a selected positive or negative angle. A latch pin 33 is able to engage in only the notch 30a that is aligned with the adjustment lock plate notch 32a. An indicator 34 and a scale 35 are provided to give a visual indication of the angle that the femoral strut 5 has been shifted relative to the tibial strut 3.

This adjustable locking arrangement is a drop lock mechanism that allows for the brace to be fixed at one of several preset positions (e.g., −10°, 0°, 10°, 20°, 30°). In some cases, the prescribing physician's protocol calls for the patient to be able to walk with the leg in a fixed position for optimum stabilization and/or to prevent post-surgical injury. The adjustable locking arrangement on both sides of the leg can be manually raised by the patient to clear the designated slot 30a in the locking plate 30 and the slot 32a in the lock plate 32. This is done by sliding the release slide 40 against the action of a return spring 42 and allows the patient to bend his/her knee (to sit down, get into a car, etc.). Once the leg is bent from the designated position, the latch pin 33 is held up against the force of return spring 42 since in now rides on the periphery of lock plate 32. When the patient stands back up, the adjustable locking arrangement automatically re-engages in the selected slot when the leg reaches the designated angular position since the return spring 42 is now free to move the latch pin 33 back into the aligned notches 30a, 32a.

Additionally, it is possible to disengage the adjustable locking arrangement, when it is desired not to require patient to ambulate with the leg in a fixed position. To do this, the release slide 40 is moved to its maximally raised position at which point a disengagement pin 45 is forced into a retaining notch 47 under the action of a disengagement spring 50. To re-engage the adjustable locking arrangement, an engagement button 52, on which disengagement pin 45 is mounted, is pressed in against the action of disengagement spring 50, thereby moving the disengagement pin 45 back out of the retaining notch 47, at which point the return spring 42 will move disengagement pin 45 back down below the retaining notch 47, and when the leg is in the appropriate position, will engage in the selected notch 30a and the notch 32a of the adjustment lock plate 32.

Another alternative feature of the present invention is can been seen in FIGS. 19 & 20. Since it may not always be easy to align the flexion stop, extension stop and lock plates 21, 23, 32, since one may move out place while another is being turned, a detent assembly can be provided to hold the plates against unintended movement.

In particular, a portion of the perimeter of the flexion stop, extension stop and lock plates 21, 23, 32 is given a corrugated or undulating contour 60 of rounded peaks and valleys. Furthermore, the retainer plate 55 is provided with spring-loaded arms 60, at the ends of which cylindrical detent pins 62 are provided which engage in the valleys of the undulating contour 60 and are able to pass over the peaks of the contour 60 as the wheels are physically turned. Instead of pins 62 which span all three rotatable plates, the pins 62 can be divided into three separate pin segments. In either case, the individual plates will be held in the position to which they are set with their respective openings axially aligned until a positive force is deliberately applied to turn one or more of the flexion stop, extension stop and lock plates 21, 23, 32.

As can be seen from the foregoing, the present invention provides a knee brace that possesses a number of features that can be used separately or together. These features allow the knee brace of the present invention to be easy and economical to produce while still being very user friendly and more comfortable to wear.

We claim:

1. A post-operative knee brace, comprising, at each of medial and lateral sides of the brace:
a tibial strut,
a femoral strut,
a joint mechanism connecting the tibial strut to the femoral strut, and
means for adjusting medial-lateral angulation of the femoral strut relative to the joint mechanism and tibial strut to match to the contour of a patient's thigh in medial-lateral directions as closely as possible.

2. Post-operative knee brace according to claim 1, wherein the means for adjusting medial-lateral angulation comprises a knurled pin retained against rotation in eyelets on a lower end of a joint connector part of the femoral strut and which also passes through a locking clamp on an upper end of a strut connector part of the joint mechanism which is adapted to be spread apart for freeing the knurled pin to rotated within the locking clamp so as to enable the angle of the femoral strut to be changed relative to the joint mechanism and tibial strut, and drawn together for holding the knurled pin tight within the clamp so that the angle of the femoral strut is fixed relative to the joint mechanism and tibial strut.

3. Post-operative knee brace according to claim 1, wherein the means for adjusting medial-lateral angulation comprises a weakened area extending crosswise of the femoral strut and which facilitates plastic deformation of the femoral strut about a centerline of the weakened area.

4. Post-operative knee brace according to claim 1, wherein the joint mechanism comprises a flexion and extension stop arrangement having a plurality of plates, one of which is an extension stop plate, another of which is a flexion stop plate, and both of which are provided with a plurality openings along a portion of their periphery and a reduced diameter portion radial stop surfaces formed at their ends, and wherein a locking lever is provided for fixing the rotational angle of the stop plates in a selected one of a plurality of positions corresponding to a respective one of said plurality of openings.

5. Post-operative knee brace according to claim 4, wherein the locking lever carries a fixing pin which is insertable into the respective one of said plurality of openings.

6. Post-operative knee brace according to claim 4, wherein a latch pin extends through said stop plates in an axial direction thereof, said latch pin engaging said stop surfaces for limiting extension and flexion to a value corresponding to the selected one of said plurality of positions.

7. Post-operative knee brace according to claim 4, wherein the locking lever has a resilient extension formed on a distal side of a pivot from a free end of the lever, the extension being shaped for holding the lever in a locked position, and also being shaped to cam inward when a lifting force is applied to the lever so as to permit the locking lever to be lifted, after which the extension is adapted to act as a leaf spring to hold the locking lever in a raised open position.

8. Post-operative knee brace according to claim 1, further comprising an adjustable locking arrangement having a locking mechanism for locking the brace in any of a number of different angular positions, said locking mechanism being releasable from a selected one of said angular positions to allow the knee to bend and being able to automatically lock back into the selected one of said angular positions.

9. Post-operative knee brace according to claim 8, wherein the locking mechanism comprises a locking plate having a plurality of peripheral notches and an adjustment lock plate having only one peripheral notch, the adjustment lock plate being rotatable for aligning said one peripheral notch with a selected one of said plurality of notches in the locking plate, and a latch pin which is engage in only the selected one of said plurality of notches in the locking plate that is aligned with the peripheral notch of the adjustment lock plate.

10. Post-operative knee brace according to claim 8, wherein the locking mechanism is a drop lock mechanism having release slide for disengaging the locking mechanism against the action of a return spring for allowing bending of a wearer's knee out of the selected one of said angular positions, the locking mechanism being adapted to automatically re-engage when the wearer's knee is returned to the selected one of said angular positions.

11. Post-operative knee brace according to claim 10, wherein the adjustable locking arrangement further comprises selectively actuatable means for holding the locking mechanism in a disengaged position and a selectively actuatable engagement means for disengaging the means for holding and re-enabling the locking mechanism.

12. A post-operative knee brace, comprising, at each of medial and lateral sides of the brace:
a tibial strut,
a femoral strut, and
a joint mechanism connecting the tibial strut to the femoral strut,
wherein the joint mechanism comprises a flexion and extension stop arrangement having a plurality of plates, one of which is an extension stop plate, another of which is a flexion stop plate, and both of which are provided with a plurality openings along a portion of their periphery and reduced diameter portion radial stop surfaces formed at their ends, and wherein a locking lever is provided for fixing the rotational angle of the stop plates in a selected one of a plurality of positions corresponding to a respective one of said plurality of openings.

13. Post-operative knee brace according to claim 12, wherein the locking lever carries a fixing pin which is insertable into the respective one of said plurality of openings.

14. Post-operative knee brace according to claim 12, wherein a latch pin extends through said stop plates in an axial direction thereof, said latch pin engaging said stop surfaces for limiting extension and flexion to a value corresponding to the selected one of said plurality of positions.

15. Post-operative knee brace according to claim 12, wherein the locking lever has a resilient extension formed on a distal side of a pivot from a free end of the lever, the extension being shaped for holding the lever in a locked position, and also being shaped to cam inward when a lifting force is applied to the lever so as to permit the locking lever to be lifted, after which the extension is adapted to act as a leaf spring to hold the locking lever in a raised open position.

16. Post-operative knee brace according to claim 12, a retainer plate is attached on an upper end of the tibial strut; and wherein the locking lever has a resilient finger which projects outward at each side, each finger having a detent projection at a free end thereof which is adapted to clip within a notch of the retainer plate for holding the lever in a locked position.

17. Post-operative knee brace according to claim 12, wherein said flexion and extension stop plates have an undulating peripheral contour, and wherein spring-loaded detents are engageable in valleys of said peripheral contour for holding the plates in position when said locking lever is disengaged.

18. A post-operative knee brace, comprising, at each of medial and lateral sides of the brace:
a tibial strut,
a femoral strut,
a joint mechanism connecting the tibial strut to the femoral strut, and
an adjustable locking arrangement having a locking mechanism for locking the brace in any of a number of different angular positions, said locking mechanism being releasable from a selected one of said angular positions to allow the knee to bend and being able to automatically lock back into the selected one of said angular positions.

19. Post-operative knee brace according to claim 18, further wherein the locking mechanism comprises a locking plate having a plurality of peripheral notches or openings and an adjustment lock plate having only one peripheral notch or opening, the adjustment lock plate being rotatable for aligning said one peripheral notch opening with a selected one of said plurality of notches or openings in the locking plate, and a latch pin which is engage in only the selected one of said plurality of notches in the locking plate that is aligned with the peripheral notch of the adjustment lock plate.

20. Post-operative knee brace according to claim 18, wherein the locking mechanism is a drop lock mechanism having release slide for disengaging the locking mechanism against the action of a return spring for allowing bending of a wearer's knee out of the selected one of said angular positions, the locking mechanism being adapted to automatically re-engage when the wearer's knee is returned to the selected one of said angular positions.

21. Post-operative knee brace according to claim 18, wherein the adjustable locking arrangement further comprises selectively actuatable means for holding the locking mechanism in a disengaged position and a selectively actuatable engagement means for disengaging the means for holding and re-enabling the locking mechanism.

22. Post-operative knee brace according to claim 19, wherein the joint mechanism further comprises a flexion and extension stop arrangement having a plurality of plates, one of which is an extension stop plate, another of which is a flexion stop plate, and both of which are provided with a plurality openings along a portion of their periphery and a reduced diameter portion radial stop surfaces formed at their ends, and wherein a locking lever is provided for fixing the rotational angle of the stop plates in a selected one of a plurality of positions corresponding to a respective one of said plurality of openings.

23. Post-operative knee brace according to claim 22, wherein the locking lever carries a fixing pin which is insertable into the respective one of said plurality of openings.

24. Post-operative knee brace according to claim 23, wherein a latch pin extends through said stop plates in an axial direction thereof, said latch pin engaging said stop surfaces for limiting extension and flexion to a value corresponding to the selected one of said plurality of positions.

25. Post-operative knee brace according to claim 19, wherein said locking plate and said flexion and extension stop plates have an undulating peripheral contour, and wherein spring-loaded detents are engageable in valleys of said peripheral contour for holding the plates in position when said locking lever is disengaged.

26. Post-operative knee brace according to claim 23, a retainer plate is attached on an upper end of the tibial strut; and wherein the locking lever has a resilient finger which projects outward at each side, each finger having a detent projection at a free end thereof which is adapted to clip within a notch of the retainer plate for holding the lever in a locked position.

27. Post-operative knee brace according to claim 18, wherein the joint mechanism further comprises a flexion and extension stop arrangement having a plurality of plates, one of which is an extension stop plate, another of which is a flexion stop plate, and both of which are provided with a plurality openings along a portion of their periphery and a reduced diameter portion radial stop surfaces formed at their ends, and wherein a locking lever is provided for fixing the rotational angle of the stop plates in a selected one of a plurality of positions corresponding to a respective one of said plurality of openings.

28. Post-operative knee brace according to claim 24, wherein said latch pin extends through said stop plates in an axial direction thereof, said latch pin engaging said stop surfaces for limiting extension and flexion to a value corresponding to the selected one of said plurality of positions.

29. Post-operative knee brace according to claim 28, a retainer plate is attached on an upper end of the tibial strut; and wherein the locking lever has a resilient finger which projects outward at each side, each finger having a detent projection at a free end thereof which is adapted to clip within a notch of the retainer plate for holding the lever in a locked position.
